(19) United States
(12) Patent Application Publication
(10) Pub. No.: US 2007/0155020 A1
(43) Pub. Date: Jul. 5, 2007

(54) Detection of chemical analytes by array of surface enhanced Raman scattering reactions

(75) Inventors: Xing Su, Cupertino, CA (US); Lei Sun, Santa Clara, CA (US); Kung-bin Sung, Seattle, WA (US)

Correspondence Address: MORRISON & FOERSTER LLP, 1650 TYSONS BOULEVARD, SUITE 400, MCLEAN, VA, 22102, US

(73) Assignee: Intel Corporation, Santa Clara, CA (US)

(21) Appl. No.: 11/305,335

(22) Filed: Dec. 19, 2005

(51) Int. Cl. G01N 33/543; G06F 19/00; C12M 1/34; C12M 3/00

(52) U.S. Cl. 436/518; 435/287.2; 702/19; 977/902

(57) ABSTRACT

A device (and methods of using and manufacturing the device) having a substrate; and a plurality of spots comprising surface enhanced Raman scattering (SERS) active particles attached to the substrate, wherein the SERS active particles reflect an incoming Raman signal to produce a reflected Raman signal having a higher intensity than that of the incoming Raman signal are disclosed. Also a device (and methods of using and manufacturing the device) a substrate; and a plurality of spots comprising composite-organic-inorganic-nanoparticles (COINs) attached to the substrate are disclosed.

### RELATED APPLICATIONS

This application is related to U.S. application Ser. No. 10/814,981 filed Mar. 30, 2004.

### FIELD OF INVENTION

The embodiments of the invention relate to arrays that include multiple sites. Each site includes surface enhanced Raman scattering (SERS) active particles or composite-organic-inorganic-nanoparticles (COINs). The embodiments also relate to quantifying the amount of an analyte in a mixture using Raman spectroscopy. The invention transcends several scientific disciplines such as polymer chemistry, biochemistry, molecular biology, medicine and medical diagnostics.

### BACKGROUND

The ability to detect and identify trace quantities of analytes has become increasingly important in virtually every scientific discipline, ranging from part per billion analyses of pollutants in sub-surface water to analysis of cancer treatment drugs in blood serum. Raman spectroscopy is one analytical technique that provides rich optical-spectral information, and surface-enhanced Raman spectroscopy (SERS) has proven to be one of the most sensitive methods for performing quantitative and qualitative analyses. A Raman spectrum, similar to an infrared spectrum, consists of a wavelength distribution of bands corresponding to molecular vibrations specific to the sample being analyzed (the analyte). In the practice of Raman spectroscopy, the beam from a light source, generally a laser, is focused upon the sample to thereby generate inelastically scattered radiation, which is optically collected and directed into a wavelength-dispersive spectrometer in which a detector converts the energy of impinging photons to electrical signal intensity.

Among many analytical techniques that can be used for chemical structure analysis, Raman spectroscopy is attractive for its capability to provide rich structure information from a small optically-focused area or detection cavity. Compared to a fluorescent spectrum that normally has a single peak with half peak width of tens of nanometers to hundreds of nanometers, a Raman spectrum has multiple bonding-structure-related peaks with half peak width of as small as a few nanometers.

Although Raman spectroscopy has proven effective for identifying certain Raman active compounds, up till now, identifying non Raman active compounds using Raman spectroscopy has not proven successful.

### BRIEF DESCRIPTION OF THE DRAWINGS

FIG. 1 shows a schematic of SERS and COIN sensor arrays.

FIG. 2 shows the Raman spectra of two related compounds and a method of determining the normalization factor that relates the spectra of one compound to the spectra of the other compound.

FIG. 3 shows the Raman spectra of aza-adenine in a variety of different solutions.

FIG. 4 shows the Raman spectra of an analyte before and after being exposed to a reducing agent.

FIG. 5 shows a graph of the Normalized Raman spectra peak height of adenine measured using SERS in the presence of H₂S.

FIG. 6 shows the Raman spectra of an analyte after being modified with a non Raman active compound.

FIG. 7 shows the Raman spectra of an analyte after being tagged with variety of different Raman active labels.

### DETAILED DESCRIPTION

As used in the specification and claims, the singular forms “a”, “an” and “the” include plural references unless the context clearly dictates otherwise. For example, the term “an array” may include a plurality of arrays unless the context clearly dictates otherwise.

An “array,” “macroarray” or “microarray” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the sample spots on the array. A macroarray generally contains sample spot sizes of about 300 microns or larger and can be easily imaged by gel and blot scanners. A microarray would generally contain spot sizes of less than 300 microns. A multiple-well array is a support that includes multiple chambers for containing sample spots.

“Solid support,” “support,” and “substrate” refer to a material or group of materials having a rigid or semi-rigid surface or surfaces. In some aspects, at least one surface of the solid support will be substantially flat, although in some aspects it may be desirable to physically separate synthesis regions for different molecules with, for example, wells, raised regions, pins, etched trenches, or the like. In certain aspects, the solid support(s) will take the form of beads, resins, gels, microspheres, or other geometric configurations.

The term “analyte”, “target” or “target molecule” refers to a molecule of interest that is to be analyzed. The analyte may be a Raman active compound or a Raman inactive compound. Further, the analyte could be an organic or inorganic molecule. Some examples of analytes may include a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to molecular probes such as chemically modified carbon nanotubes, carbon nanotube bundles, nanowires, nanoclusters or nanoparticles. The analyte molecule may be fluorescently labeled DNA or RNA.

The term “probe” or “probe molecule” refers to a molecule that binds to a target molecule for the analysis of the target. The probe or probe molecule is generally, but not necessarily, has a known molecular structure or sequence. The probe or probe molecule is generally, but not necessarily, attached to the substrate of the array. The probe or probe molecule is typically a nucleotide, an oligonucleotide, or a protein, including, for example, cDNA or pre-synthesized polynucleotide deposited on the array. Probes molecules are biomolecules capable of undergoing binding or molecular recognition events with target molecules. (In some references, the terms “target” and “probe” are defined opposite to the definitions provided here.) The polynucleotide probes require only the sequence information of genes, and thereby can exploit the genome sequences of an organism. In cDNA arrays, there could be cross-hybridization due to sequence homologies among members of a gene family. Polynucleotide arrays can be specifically designed to differentiate between highly homologous members of a gene family as well as spliced forms of the same gene (exon-specific). Polynucleotide arrays of the embodiment of this invention could also be designed to allow detection of mutations and single nucleotide polymorphism. A probe or probe molecule can be a capture molecule.

The term “bi-functional linker group” refers to an organic chemical compound that has at least two chemical groups or moieties, such are, carboxyl group, amine group, thiol group, aldehyde group, epoxy group, that can be covalently modified specifically; the distance between these groups is equivalent to or greater than 5-carbon bonds.

The term “capture molecule” refers to a molecule that is immobilized on a surface. The capture molecule is generally, but not necessarily, binds to a target or target molecule. The capture molecule is typically a nucleotide, an oligonucleotide, or a protein, but could also be a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to a target molecule that is bound to a probe molecule to form a complex of the capture molecule, target molecule and the probe molecule. The capture molecule may be fluorescently labeled DNA or RNA. The capture molecule may or may not be capable of binding to just the target molecule or just the probe molecule.

The terms “die,” “polymer array chip,” “DNA array,” “array chip,” “DNA array chip,” or “bio-chip” are used interchangeably and refer to a collection of a large number of probes arranged on a shared substrate which could be a portion of a silicon wafer, a nylon strip or a glass slide.

The term “chip” or “microchip” refers to a microelectronic device made of semiconductor material and having one or more integrated circuits or one or more devices. A “chip” or “microchip” is typically a section of a wafer and made by slicing the wafer. A “chip” or “microchip” may comprise many miniature transistors and other electronic components on a single thin rectangle of silicon, sapphire, germanium, silicon nitride, silicon germanium, or of any other semiconductor material. A microchip can contain dozens, hundreds, or millions of electronic components.

The term “molecule” generally refers to a macromolecule or polymer as described herein. However, arrays comprising single molecules, as opposed to macromolecules or polymers, are also within the scope of the embodiments of the invention.

“Predefined region” or “spot” or “pad” refers to a localized area on a solid support. The spot could be intended to be used for formation of a selected molecule and is otherwise referred to herein in the alternative as a “selected” region. The spot may have any convenient shape, e.g., circular, rectangular, elliptical, wedge-shaped, etc. For the sake of brevity herein, “predefined regions” are sometimes referred to simply as “regions” or “spots.” In some embodiments, a predefined region and, therefore, the area upon which each distinct molecule is synthesized is smaller than about 1 cm² or less than 1 mm², and still more preferably less than 0.5 mm². In most preferred embodiments the regions have an area less than about 10,000 μm² or, more preferably, less than 100 μm², and even more preferably less than 10 μm² or less than 1 μm². Additionally, multiple copies of the polymer will typically be synthesized within any preselected region. The number of copies can be in the hundreds to the millions. A spot could contain an electrode to generate an electrochemical reagent, a working electrode to synthesize a polymer and a confinement electrode to confine the generated electrochemical reagent. The electrode to generate the electrochemical reagent could be of any shape, including, for example, circular, flat disk shaped and hemisphere shaped.

“Micro-Electro-Mechanical Systems (MEMS)” is the integration of mechanical elements, sensors, actuators, and electronics on a common silicon substrate through microfabrication technology. While the electronics are fabricated using integrated circuit (IC) process sequences (e.g., CMOS, Bipolar, or BICMOS processes), the micromechanical components could be fabricated using compatible “micromachining” processes that selectively etch away parts of the silicon wafer or add new structural layers to form the mechanical and electromechanical devices. Microelectronic integrated circuits can be thought of as the “brains” of a system and MEMS augments this decision-making capability with “eyes” and “arms”, to allow microsystems to sense and control the environment. Sensors gather information from the environment through measuring mechanical, thermal, biological, chemical, optical, and magnetic phenomena. The electronics then process the information derived from the sensors and through some decision making capability direct the actuators to respond by moving, positioning, regulating, pumping, and filtering, thereby controlling the environment for some desired outcome or purpose. Because MEMS devices are manufactured using batch fabrication techniques similar to those used for integrated circuits, unprecedented levels of functionality, reliability, and sophistication can be placed on a small silicon chip at a relatively low cost.

“Microprocessor” is a processor on an integrated circuit (IC) chip. The processor may be one or more processor on one or more IC chip. The chip is typically a silicon chip with thousands of electronic components that serves as a central processing unit (CPU) of a computer or a computing device.

A “macromolecule” or “polymer” comprises two or more monomers covalently joined. The monomers may be joined one at a time or in strings of multiple monomers, ordinarily known as “oligomers.” Thus, for example, one monomer and a string of five monomers may be joined to form a macromolecule or polymer of six monomers. Similarly, a string of fifty monomers may be joined with a string of hundred monomers to form a macromolecule or polymer of one hundred and fifty monomers. The term polymer as used herein includes, for example, both linear and cyclic polymers of nucleic acids, polynucleotides, polynucleotides, polysaccharides, oligosaccharides, proteins, polypeptides, peptides, phospholipids and peptide nucleic acids (PNAs). The peptides include those peptides having either α-, β-, or co-amino acids. In addition, polymers include heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure.

A “nanomaterial” as used herein refers to a structure, a device or a system having a dimension at the atomic, molecular or macromolecular levels, in the length scale of approximately 1-100 nanometer range. Preferably, a nanomaterial has properties and functions because of the size and can be manipulated and controlled on the atomic level.

A “carbon nanotube” refers to a fullerene molecule having a cylindrical or toroidal shape. A “fullerene” refers to a form of carbon having a large molecule consisting of an empty cage of sixty or more carbon atoms.

The term “nucleotide” includes deoxynucleotides and analogs thereof. These analogs are those molecules having some structural features in common with a naturally occurring nucleotide such that when incorporated into a polynucleotide sequence, they allow hybridization with a complementary polynucleotide in solution. Typically, these analogs are derived from naturally occurring nucleotides by replacing and/or modifying the base, the ribose or the phosphodiester moiety. The changes can be tailor-made to stabilize or destabilize hybrid formation, or to enhance the specificity of hybridization with a complementary polynucleotide sequence as desired, or to enhance stability of the polynucleotide.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers.

An “oligonucleotide” is a polynucleotide having 2 to 20 nucleotides. Analogs also include protected and/or modified monomers as are conventionally used in polynucleotide synthesis. As one of skill in the art is well aware, polynucleotide synthesis uses a variety of base-protected nucleoside derivatives in which one or more of the nitrogens of the purine and pyrimidine moiety are protected by groups such as dimethoxytrityl, benzyl, tert-butyl, isobutyl and the like.

For instance, structural groups are optionally added to the ribose or base of a nucleoside for incorporation into a polynucleotide, such as a methyl, propyl or allyl group at the 2′-O position on the ribose, or a fluoro group which substitutes for the 2′-O group, or a bromo group on the ribonucleoside base. 2′-O-methyloligoribonucleotides (2′-O-MeORNs) have a higher affinity for complementary polynucleotides (especially RNA) than their unmodified counterparts. Alternatively, deazapurines and deazapyrimidines in which one or more N atoms of the purine or pyrimidine heterocyclic ring are replaced by C atoms can also be used.

The phosphodiester linkage, or “sugar-phosphate backbone” of the polynucleotide can also be substituted or modified, for instance with methyl phosphonates, O-methyl phosphates or phosphororthioates. Another example of a polynucleotide comprising such modified linkages for purposes of this disclosure includes “peptide polynucleotides” in which a polyamide backbone is attached to polynucleotide bases, or modified polynucleotide bases. Peptide polynucleotides which comprise a polyamide backbone and the bases found in naturally occurring nucleotides are commercially available.

Nucleotides with modified bases can also be used in the embodiments of the invention. Some examples of base modifications include 2-aminoadenine, 5-methylcytosine, 5-(propyn-1-yl)cytosine, 5-(propyn-1-yl)uracil, 5-bromouracil, 5-bromocytosine, hydroxymethylcytosine, methyluracil, hydroxymethyluracil, and dihydroxypentyluracil which can be incorporated into polynucleotides in order to modify binding affinity for complementary polynucleotides.

Groups can also be linked to various positions on the nucleoside sugar ring or on the purine or pyrimidine rings which may stabilize the duplex by electrostatic interactions with the negatively charged phosphate backbone, or through interactions in the major and minor groves. For example, adenosine and guanosine nucleotides can be substituted at the N² position with an imidazolyl propyl group, increasing duplex stability. Universal base analogues such as 3-nitropyrrole and 5-nitroindole can also be included. A variety of modified polynucleotides suitable for use in the embodiments of the invention are described in the literature.

When the macromolecule of interest is a peptide, the amino acids can be any amino acids, including α, β, or ω-amino acids. When the amino acids are α-amino acids, either the L-optical isomer or the D-optical isomer may be used. Additionally, unnatural amino acids, for example, β-alanine, phenylglycine and homoarginine are also contemplated by the embodiments of the invention. These amino acids are well-known in the art.

A “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often more than 20 amino acid monomers long.

A “protein” is a long polymer of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies.

The term “sequence” refers to the particular ordering of monomers within a macromolecule and it may be referred to herein as the sequence of the macromolecule.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” For example, hybridization refers to the formation of hybrids between a probe polynucleotide (e.g., a polynucleotide of the invention which may include substitutions, deletion, and/or additions) and a specific target polynucleotide (e.g., an analyte polynucleotide) wherein the probe preferentially hybridizes to the specific target polynucleotide and substantially does not hybridize to polynucleotides consisting of sequences which are not substantially complementary to the target polynucleotide. However, it will be recognized by those of skill that the minimum length of a polynucleotide desired for specific hybridization to a target polynucleotide will depend on several factors: G/C content, positioning of mismatched bases (if any), degree of uniqueness of the sequence as compared to the population of target polynucleotides, and chemical nature of the polynucleotide (e.g., methylphosphonate backbone, phosphorothiolate, etc.), among others.

Methods for conducting polynucleotide hybridization assays have been well developed in the art. Hybridization assay procedures and conditions will vary depending on the application and are selected in accordance with the general binding methods known in the art.

It is appreciated that the ability of two single stranded polynucleotides to hybridize will depend upon factors such as their degree of complementarity as well as the stringency of the hybridization reaction conditions.

As used herein, “stringency” refers to the conditions of a hybridization reaction that influence the degree to which polynucleotides hybridize. Stringent conditions can be selected that allow polynucleotide duplexes to be distinguished based on their degree of mismatch. High stringency is correlated with a lower probability for the formation of a duplex containing mismatched bases. Thus, the higher the stringency, the greater the probability that two single-stranded polynucleotides, capable of forming a mismatched duplex, will remain single-stranded. Conversely, at lower stringency, the probability of formation of a mismatched duplex is increased.

The appropriate stringency that will allow selection of a perfectly-matched duplex, compared to a duplex containing one or more mismatches (or that will allow selection of a particular mismatched duplex compared to a duplex with a higher degree of mismatch) is generally determined empirically. Means for adjusting the stringency of a hybridization reaction are well-known to those of skill in the art.

A “ligand” is a molecule that is recognized by a particular receptor. Examples of ligands that can be investigated by this invention include, but are not restricted to, agonists and antagonists for cell membrane receptors, toxins and venoms, viral epitopes, hormones, hormone receptors, peptides, enzymes, enzyme substrates, cofactors, drugs (e.g. opiates, steroids, etc.), lectins, sugars, polynucleotides, nucleic acids, oligosaccharides, proteins, and monoclonal antibodies.

A “receptor” is molecule that has an affinity for a given ligand. Receptors may-be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term “receptors” is used herein, no difference in meaning is intended. A “Ligand Receptor Pair” is formed when two macromolecules have combined through molecular recognition to form a complex. Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value would be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies: For instance, the invention may be useful in investigating the ligand-binding site on the antibody molecule which combines with the epitope of an antigen of interest; determining a sequence that mimics an antigenic epitope may lead to the-development of vaccines of which the immunogen is based on one or more of such sequences or lead to the development of related diagnostic agents or compounds useful in therapeutic treatments such as for auto-immune diseases (e.g., by blocking the binding of the “anti-self” antibodies).

d) Nucleic Acids: Sequences of nucleic acids may be synthesized to establish DNA or RNA binding sequences.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant.

f) Hormone receptors: Examples of hormones receptors include, e.g., the receptors for insulin and growth hormone. Determination of the ligands which bind with high affinity to a receptor is useful in the development of, for example, an oral replacement of the daily injections which diabetics take to relieve the symptoms of diabetes. Other examples are the vasoconstrictive hormone receptors; determination of those ligands which bind to a receptor may lead to the development of drugs to control blood pressure.

g) Opiate receptors: Determination of ligands which bind to the opiate receptors in the brain is useful in the development of less-addictive replacements for morphine and related drugs.

The phrase “SERS active particle refers” to particles that produce the surface-enhanced Raman scattering effect. The SERS active particles generate surface enhanced Raman signal specific to the analyte molecules when the analyte-SERS complexes are excited with a light source. The enhanced Raman scattering effect provides a greatly enhanced Raman signal from Raman-active analyte molecules that have been adsorbed onto certain specially-prepared SERS active particle surfaces. Typically, the SERS active particle surfaces are metal surfaces. Increases in the intensity of Raman signal have been regularly observed on the order of 10⁴-10¹⁴ for some systems. SERS active particles include a variety of metals including coinage (Au, Ag, Cu), alkalis (Li, Na, K), Al, Pd and Pt.

The term “COIN” refers to a composite-organic-inorganic nanocluster(s)/nanoparticle(s). The COIN could be surface-enhanced Raman scattering (SERS, also referred to as surface-enhanced Raman spectroscopy)-active nanoclusters incorporated into a gel matrix and used in certain other analyte separation techniques described herein. COINs are composite organic-inorganic nanoclusters. These SERS-active probe constructs comprise a core and a surface, wherein the core comprises a metallic colloid comprising a first metal and a Raman-active organic compound. The COINs can further comprise a second metal different from the first metal, wherein the second metal forms a layer overlying the surface of the nanoparticle. The COINs can further comprise an organic layer overlying the metal layer, which organic layer comprises the probe. Suitable probes for attachment to the surface of the SERS-active nanoclusters include, without limitation, antibodies, antigens, polynucleotides, oligonucleotides, receptors, ligands, and the like.

The metal required for achieving a suitable SERS signal is inherent in the COIN, and a wide variety of Raman-active organic compounds can be incorporated into the particle. Indeed, a large number of unique Raman signatures can be created by employing nanoclusters containing Raman-active organic compounds of different structures, mixtures, and ratios. Thus, the methods described herein employing COINs are useful for the simultaneous detection of many analytes in a sample, resulting in rapid qualitative analysis of the contents of “profile” of a body fluid.

COINs could be prepared using standard metal colloid chemistry. The preparation of COINs also takes advantage of the ability of metals to adsorb organic compounds. Indeed, since Raman-active organic compounds are adsorbed onto the metal during formation of the metallic colloids, many Raman-active organic compounds can be incorporated into the COIN without requiring special attachment chemistry.

In general, the COINs could be prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. COINs of different sizes can be enriched by centrifugation.

The COINs can include a second metal different from the first metal wherein the second metal forms a layer overlying the surface of the nanoparticle. To prepare this type of SERS-active nanoparticle, COINs are placed in an aqueous solution containing suitable second metal cations and a reducing agent. The components of the solution are then subject to conditions that reduce the second metallic cations so as to form a metallic layer overlying the surface of the nanoparticle. In certain embodiments, the second metal layer includes metals, such as, for example, silver, gold, platinum, aluminum, and the like. Typically, COINs are clustered structures and range in size from about 50 nm to 100 nm.

Typically, organic compounds are attached to a layer of a second metal in COINs by covalently attaching organic compounds to the surface of the metal layer Covalent attachment of an organic layer to the metallic layer can be achieved in a variety ways well known to those skilled in the art, such as for example, through thiol-metal bonds. In alternative embodiments, the organic molecules attached to the metal layer can be crosslinked to form a molecular network.

The COIN(s) can include cores containing magnetic materials, such as, for example, iron oxides, and the like such that the COIN is a magnetic COIN. Magnetic COINs can be handled without centrifugation using commonly available magnetic particle handling systems. Indeed, magnetism can be used as a mechanism for separating biological targets attached to magnetic COIN particles tagged with particular biological probes.

As used herein, “Raman-active organic compound” refers to an organic molecule that produces a unique SERS signature in response to excitation by a laser. A variety of Raman-active organic compounds are contemplated for use as components in COINs. In certain embodiments, Raman-active organic compounds are polycyclic aromatic or heteroaromatic compounds. Typically the Raman-active organic compound has a molecular weight less than about 300 Daltons.

Additional, non-limiting examples of Raman-active organic compounds useful in COINs include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins, aminoacridine, and the like.

In certain embodiments, the Raman-active compound is adenine, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, or 9-amino-acridine 4-amino-pyrazolo(3,4-d)pyrimidine, rhodamine 6G, rhodamine B, crystal violet, basic fuchsin, cyanine 2, cyanine 3, or 2-fluoroadenine. In one embodiment, the Raman-active compound is adenine.

When “fluorescent compounds” are incorporated into COINs, the fluorescent compounds can include, but are not limited to, dyes, intrinsically fluorescent proteins, lanthanide phosphors, and the like. Dyes useful for incorporation into COINs include, for example, rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS), Lucifer Yellow, IAEDANS, 7-Me₂, N-coumarin-4-acetate, 7-OH-4-CH₃-coumarin-3-acetate, 7-NH₂-4CH₃-coumarin-3-acetate (AMCA), monobromobimane, pyrene trisulfonates, such as Cascade Blue, and monobromotrimethyl-ammoniobimane.

Multiplex testing of a complex sample would generally be based on a coding system that possesses identifiers for a large number of reactants in the sample. The primary variable that determines the achievable numbers of identifiers in currently known coding systems is, however, the physical dimension. Techniques, based on surface-enhanced Raman scattering (SERS) of organic compounds, could be used in the embodiments of this invention for developing chemical structure-based coding systems. The organic compound-assisted metal fusion (OCAM) method could be used to produce composite organic-inorganic nanoparticles (COIN) that are highly effective in generating SERS signals allows synthesis of COIN labels from a wide range of organic compounds to produce sufficient distinguishable COIN Raman signatures to assay any complex biological sample. Thus COIN particles may be used as a coding system for multiplex and amplification-free detection of bioanalytes at near single molecule levels.

COIN particles generate intrinsic SERS signal without additional reagents. Using the OCAMF-based COIN synthesis chemistry, it is possible to generate a large number of different COIN signatures by mixing a limited number of Raman labels for use in multiplex assays in different ratios and combinations. In a simplified scenario, the Raman spectrum of a sample labeled with COIN particles may be characterized by three parameters: (a) peak position (designated as L), which depends on the chemical structure of Raman labels used and the umber of available labels, (b) peak number (designated as M), which depends on the number of labels used together in a single COIN, and (c) peak height (designated as i), which depends on the ranges of relative peak intensity.

The total number of possible distinguishable Raman signatures (designated as T) may be calculated from the following equation:

$$T = {\sum }_{k = 1}^{M} \frac{L !}{( L - k ) ! k !} P ( i , k )$$

where P(i, k)=i^{k}−i+1, being the intensity multiplier which represents the number of distinct Raman spectra that may be generated by combining k (k=1 to M) labels for a given i value. The multiple organic compounds may be mixed in various combinations, numbers and ratios to make the multiple distinguishable Raman signatures. It has been shown that spectral signatures having closely positioned peaks (15 cm⁻¹) may be resolved visually. Theoretically, over a million of Raman signatures may be made within the Raman shift range of 500-2000 cm⁻¹ by incorporating multiple organic molecules into COIN as Raman labels using the OCAMF-based COIN synthesis chemistry.

Thus, OCAMF chemistry allows incorporation of a wide range of Raman labels into metal colloids to perform parallel synthesis of a large number of COIN labels with distinguishable Raman signatures in a matter of hours by mixing several organic Raman-active compounds of different structures, mixtures, and ratios for use in the invention methods described herein.

COINs may be used to detect the presence of a particular target analyte, for example, a nucleic acid, oligonucleotide, protein, enzyme, antibody or antigen. The nanoclusters may also be used to screen bioactive agents, i.e. drug candidates, for binding to a particular target or to detect agents like pollutants. Any analyte for which a probe moiety, such as a peptide, protein, oligonucleotide or aptamer, may be designed can be used in combination with the disclosed nanoclusters.

Also, SERS-active COINs that have an antibody as binding partner could be used to detect interaction of the Raman-active antibody labeled constructs with antigens either in solution or on a solid support. It will be understood that such immunoassays can be performed using known methods such as are used, for example, in ELISA assays, Western blotting, or protein arrays, utilizing a SERS-active COIN having an antibody as the probe and acting as either a primary or a secondary antibody, in place of a primary or secondary antibody labeled with an enzyme or a radioactive compound. In another example, a SERS-active COIN is attached to an enzyme probe for use in detecting interaction of the enzyme with a substrate.

Another group of exemplary methods could use the SERS-active COINs to detect a target nucleic acid. Such a method is useful, for example, for detection of infectious agents within a clinical sample, detection of an amplification product derived from genomic DNA or RNA or message RNA, or detection of a gene (cDNA) insert within a clone. For certain methods aimed at detection of a target polynucleotide, an oligonucleotide probe is synthesized using methods known in the art. The oligonucleotide is then used to functionalize a SERS-active COIN. Detection of the specific Raman label in the SERS-active COIN identifies the nucleotide sequence of the oligonucleotide probe, which in turn provides information regarding the nucleotide sequence of the target polynucleotide.

The term “complementary” refers to the topological compatibility or matching together of interacting surfaces of a ligand molecule and its receptor. Thus, the receptor and its ligand can be described as complementary, and furthermore, the contact surface characteristics are complementary to each other.

The term “waveguide” refers to a device that controls the propagation of an electromagnetic wave so that the wave is forced to follow a path defined by the physical structure of the guide. Generally speaking, the electric and magnetic fields of an electromagnetic wave have a number of possible arrangements when the wave is traveling through a waveguide. Each of these arrangements is known as a mode of propagation. Optical waveguides are used at optical frequencies. An “optical waveguide” is any structure having the ability to guide optical energy. Optical waveguides may be (a) thin-film deposits used in integrated optical circuits (IOCs) or (b) optical fibers.

The term “optical switch” refers to a switch that enables signals in optical fibers or integrated optical circuits (IOCs) to be selectively switched from one circuit to another. An optical switch may operate by (a) mechanical means, such as physically shifting an optical fiber to drive one or more alternative fibers, or (b) electro-optic effects, magneto-optic effects, or other methods. Slow optical switches, such as those using moving fibers, may be used for alternate routing of an optical transmission path. Fast optical switches, such as those using electro-optic or magneto-optic effects, may be used to perform logic operations. One type of an optical switch is a thin film optical switch, which is a switch having multilayered films of material of different optical characteristics, that is capable of switching transmitted light by using electro-optic, electro-acoustic, or magneto-optic effects to obtain signal switching, and is usually used as a component in integrated optical circuits. Thin-film optical switches may support only one propagation mode.

The term “PIN diode” refers to positive-intrinsic-negative diode. A photodiode with a large, neutrally doped intrinsic region sandwiched between p-doped and n-doped semiconducting regions. A PIN diode exhibits an increase in its electrical conductivity as a function of the intensity, wavelength, and modulation rate of the incident radiation. A PIN diode is also called photodiode.

The terms “spectrum” or “spectra” refer to the intensities of electromagnetic radiation as a function of wavelength or other equivalent units, such as wavenumber, frequency, and energy level.

The term “spectrometer” refers to an instrument equipped with scales for measuring wavelengths or indexes of refraction.

The term “dispersive spectrometer” refers to a spectrometer that generates spectra by optically dispersing the incoming radiation into its frequency or spectral components. Dispersive spectrometers can be further classified into two types: monochromators and spectrographs. A monochromator uses a single detector, narrow slit(s) (usually two, one at the entrance and another at the exit port), and a rotating dispersive element allowing the user to observe a selected range of wavelength. A spectrograph, on the other hand, uses an array of detector elements and a stationary dispersive element. In this spectral elements over a wide range of wavelengths are obtained at the same time, therefore providing faster measurements with a more expensive detection system.

The term “dispersive element” refers to a component of a dispersive spectrometer that can disperse electromagnetic radiation such a light. Dispersive elements include prisms and gratings.

The term “interferometer” refers to an instrument that uses the principle of interference of electromagnetic waves for purposes of measurement. For example, it could be any of several optical, acoustic, or radio frequency instruments that use interference phenomena between a reference wave and an experimental wave or between two parts of an experimental wave to determine wavelengths and wave velocities, measure very small distances and thicknesses, and calculate indices of refraction.

The term “non-dispersive element” refers to an interferometer that does not disperse electromagnetic radiation in spatial domain but instead creates a phase shift in the electromagnetic radiation.

The term “Fourier transform spectrometer” refers to a spectrometer used for Fourier transform spectroscopy, which is a measurement technique whereby spectra are collected based on the response from a pulse of electromagnetic radiation. It can be applied to variety of types of spectroscopy including infrared spectroscopy (FTIR), nuclear magnetic resonance, and electron spin resonance spectroscopy. Fourier transform spectroscopy can be more sensitive and has a much shorter sampling time than conventional spectroscopic techniques. For example, in a conventional (or “continuous wave”) nucleic magnetic resonance spectrometer, a sample is exposed to electromagnetic radiation and the response (usually the intensity of transmitted radiation) is monitored. The energy of the radiation is varied over the desired range and the response is plotted as a function of radiation energy (or frequency). At certain resonant frequencies characteristic of the specific sample, the radiation will be absorbed resulting in a series of peaks in the spectrum, which can then be used to identify the sample. (In magnetic spectroscopy, the magnetic field is often varied instead of the frequency of the incident radiation, though the spectra are effectively the same as if the field had been kept constant and the frequency varied. This is largely a question of experimental convenience.) Instead of varying the energy of the electromagnetic radiation, Fourier Transform nucleic magnetic resonance spectroscopy exposes the sample to a single pulse of radiation and measures the response. The resulting signal, called a free induction decay, contains a rapidly decaying composite of all possible frequencies. Due to resonance by the sample, resonant frequencies will be dominant in the signal and by performing a mathematical operation called a Fourier transform on the signal the frequency response can be calculated. In this way the Fourier transform nucleic magnetic resonance spectrometer can produce the same kind of spectrum as a conventional spectrometer, but generally in a much shorter time.

The term “optical bench” refers to an apparatus for observation and measurement of optical phenomena. For example, it could be an apparatus such as a special table or rigid beam, for the precise positioning of light sources, screens, and optical instruments used for optical and photometric studies, having a ruled bar to which these devices can be attached and along which they can be readily adjusted.

The term “interferogram” or “Fourier transform spectrum” used herein means the detector response as a function of the optical path length difference caused by the interference of electromagnetic radiation.

Embodiments of this invention relate to an array of SERS active particles. The SERS active particles can be COIN particles or other SERS active particles such as silver aggregates. The array of SERS active particles can be used to identify and/or quantify a variety of Raman active and also non Raman active analytes.

One embodiment of this invention is a device including a substrate and a plurality of spots comprising surface enhanced Raman scattering (SERS) active particles attached to the substrate. The SERS active particles generate surface enhanced Raman signal specific to the analyte molecules when the analyte-SERS complexes are excited with a light source.

Preferably, the SERS active particles comprise a metal. Preferred metals include gold, silver, copper, lithium, sodium, potassium, palladium, platinum, and aluminum. The SERS active particles may also preferably include composite-organic-inorganic-nanoparticles (COINs). Preferred COINs include adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, rhodamine 6G, rhodamine B, crystal violet, basic fuchsin, cyanine 2, cyanine 3, and 9-amino-acridine.

Preferably, the composition of the SERS active particles included a first spot has a different composition than the SERS active particles comprising at least one other spot. Alternatively, or in addition, preferably the concentration of the SERS active particles comprising a first spot have a different concentration than the SERS active particles comprising at least one other spot.

Preferably, the device also includes a Raman spectrometer. Preferably, the substrate of the device includes a multiple-well array or a surface comprising a plurality of sub-surfaces. Preferably, the SERS active particles are attached to the substrate through thiol groups.

In another embodiment, the device includes a substrate and a plurality of spots including composite-organic-inorganic-nanoparticles (COINs) attached to the substrate. Preferably, the COINs are attached to the substrate through bi-functional linker groups.

Yet another embodiment is a method of quantifying an analyte in a mixture. The method includes determining a normalization equation that correlates a Raman spectra of a first analyte to a Raman spectra of a second analyte for a given analyte concentration, obtaining the concentration of the first analyte in a mixture, measuring the Raman spectra of the first analyte in the mixture and a Raman spectra of the second analyte in the mixture, and determining the concentration of the second analyte in the mixture utilizing the normalization equation.

Preferably, the normalization equation is determined by obtaining a Raman spectra of the first analyte across a range of concentration levels, obtaining a Raman spectra of the second analyte across a range of concentrations, and correlating the Raman spectra of the first analyte to the Raman spectra of the second analyte to determine the normalization equation.

Preferably, the Raman spectra of the first and second analytes in the mixture were obtained utilizing surface enhanced Raman scattering (SERS) active particles. Preferred SERS particles include composite-organic-inorganic-nanoparticles (COINs). Preferably, the SERS active particles include gold, silver, copper, lithium, sodium, potassium, palladium, platinum, or aluminum.

Preferably, the first analyte and the second analyte are related in terms of molecular backbone structure, with difference in side groups or difference in configuration of the side groups with respect to the backbone structure. Preferably, the first and second analytes are both organic compounds or are both inorganic compounds.

The normalization equation may be linear or non-linear for a range of concentrations. The mixture may include a third analyte.

Preferably, Raman active labels may be attached to the first and second analytes. One or more of the analytes may not be a Raman active compound and of this analyte may be created by modifying the Raman spectra of a Raman active compound. Accordingly, the Raman spectra of the first analyte may be created by modifying the Raman spectra of a Raman active compound.

Another embodiment is a method of identifying an analyte in a mixture. The method includes obtaining a Raman spectra of a series of known substances in one or more environments, measuring a Raman spectra of an analyte in a mixture, and comparing the Raman spectra of the analyte in the mixture to the Raman spectra of the series of known substances in one or more environments to identify the analyte in the mixture.

Preferably, the Raman spectra of the series of known substances in one or more environments are obtained by measuring the Raman spectra of the known substances in one or more environments. Preferably, the mixture includes water, ethanol or polysorbate 20.

Preferably, the mixture includes a reducing agent. Preferably, one or more components in the mixture reacts with the analyte. The Raman spectra of the analyte in the mixture is measured using surface enhanced Raman scattering (SERS) active particles.

This method may also include identifying one or more components in the mixture besides the analyte. In addition, the Raman spectra of the series of known substances in one or more environments may be obtained for a plurality of known substance concentrations.

Yet another embodiment is a method of quantifying an analyte in a solution. The method includes obtaining a Raman spectra of a known substance in one or more environments at a plurality of different concentrations, measuring a Raman spectra of an analyte in a mixture, and comparing the Raman spectra of the analyte in the mixture to the Raman spectra of a known substance in one or more environments at a plurality of different concentrations to determine the concentration of the analyte in the mixture.

Preferably, the Raman spectra of a known substance in one or more environments at a plurality of different concentrations are obtained by measuring the Raman spectra of the known substance in one or more environments.

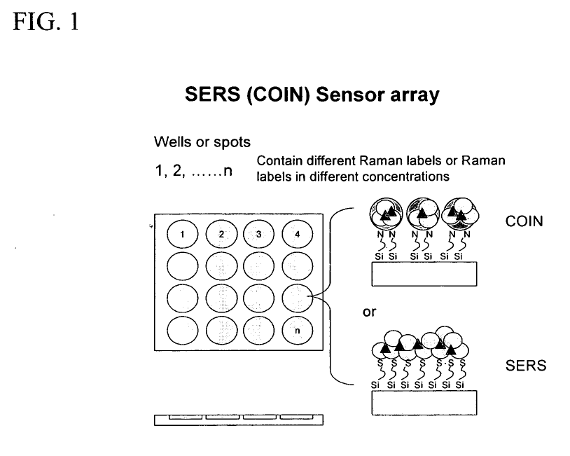

FIG. 1 shows an array of SERS active particles. The array includes a plurality of spots attached to a substrate. Each of the spots may be the same or may include a different composition and/or concentration of SERS active particles. The array can be a multiple-well array or a surface containing multiple sub-surfaces. For a multiple well array, serial dilutions of a standard compound can be used to calibrate the concentration of the analyte.

The concentration of the analyte can be determined by comparing the peak ratios of known reference compounds over a variety of concentrations to the actual peak ratio of the analyte. Accurate identification of the analyte can be accomplished utilizing the analyte's Raman spectra. Once the analyte is identified, similar reference compounds can be determined for the comparison.

Preferably a library of peak is maintained for a variety of reference compounds over a range of concentrations. Instead of reference compounds, the analyte peak ratios can be directly compared to the known peak ratios for the analyte at a variety of concentrations if these peak ratios are available.

For a sub-surface array, an analyte is exposed to various SERS sites or COINs formed with different Raman labels, with which the analyte interacts differently. Thus the signal patterns (spectral shapes or intensities) are used for analyte identification. A Raman spectrometer and related software are part of the detection system.

In one embodiment, the SERS active particles are COIN particles. The COIN particles may include Raman-active compounds. Each spot on the array may include the same COIN particles. Alternatively, each spot in the array may include different COIN particles. For example each spot may employ COINs having Raman active compounds having different structures, mixtures, and concentrations.

The array of COIN particles can be used to identify both Raman active and non Raman active analytes. Non Raman active compounds can be identified using the COIN array because these compounds can interact with one or more of the COINs in the array or otherwise alter the COIN signatures. Since different compounds may interact with different COIN particles, by having different spots contain different COIN particles, a sample containing an analyte can be tested for its interaction with several different COIN particles simultaneously.

The largest Raman signature changes can be achieved when the analyte chemically reacts and binds to the COIN particles. A variety of constructs can be included in the COINs to facilitate the interaction of the COIN and the analyte. For example, a probe moiety can be used in conjunction with the COIN particles to initiate binding and detection of complex molecules such as peptides, proteins, oligonucleotides or aptamer.

In addition, the claimed process may or may not involve an analyte chemically binding to the COIN particles for the analyte to change the Raman signature produced by the COIN. This is because there are several ways for the analytes to change the COIN signature, for example: 1) it may react with the organic Raman label compound in the COIN (reduction or oxidation), 2) it may change the interaction of the Raman label molecules with the silver particles (orientation, enhancing or decreasing the binding or some functional groups to silver, and 3) it may insert into the COIN and thus create a more complex or simpler COIN signature.

The COIN array can be made, for example, by the following contact printing methods as used for DNA or protein microarray fabrication since COIN particles are nano size dimension and thus remain in solution in colloidal state. In addition, COIN arrays may also be made by non-contact printing methods, similar to inject printing method, where print heads are filled with different COIN solutions. After delivering COIN particles onto a solid support substrate, COIN particles can be immobilized by chemical cross-linking through functional groups on the COIN surface and the substrate surface. The COIN particles can also be attached to the substrate utilizing bi-functional linkers.

In another embodiment the SERS array includes surface enhanced Raman scattering active particles that do not contain Raman-labels. For example, gold silver, platinum copper or aluminum particles can be placed in the array to enhance the Raman spectra of Raman active analytes. Silver colloidal particles have been found to be particularly useful for SERS arrays. Since these SERS active particles do not themselves produce the detected Raman spectra, the analyte must produce a detectable Raman Spectra.

Furthermore, surface enhanced Raman scattering (SERS) techniques make it possible to obtain many-fold Raman signal enhancement, for example, by about 10 to about 10000 fold increase, more preferably, about 100 to about 1000 fold increase. Such huge enhancement factors could be attributed primarily to enhanced electromagnetic fields on curved surfaces of coinage metals. Although the electromagnetic enhancement (EME) has been shown to be related to the roughness of metal surfaces or particle size when individual metal colloids are used, SERS is most effectively detected from aggregated colloids. For example, chemical enhancement can also be obtained by placing molecules in a close proximity to the surface in certain orientations.

Embodiments of this invention also relate to quantifying the amount of a compound using Raman spectroscopy. One of the problems with traditional Raman spectroscopy is that although it can be used to identify a variety of substances, Raman spectroscopy has traditionally been unable to quantify the concentration of the substances in a mixture. This is because the Raman spectrum of an analyte is not directly related to the concentration of the analyte in the mixture.

Raman spectroscopy, however, can be used to quantify the amount of a compound in a mixture if the amount of a related compound is already known. This can be accomplished using “Competitive SERS.” In Competitive SERS the Raman spectra of at least a first analyte and a second analyte are recorded for a series of concentrations. A library can even be produced containing the concentrations of several analytes across a variety of different concentrations.

Other than the concentration of the analytes, preferably the rest of the testing conditions remain as constant as possible. The testing conditions can include the measurement procedure, conditions and instrument such as Raman spectrometer, the buffer conditions and solution in which the analyte is analyzed.

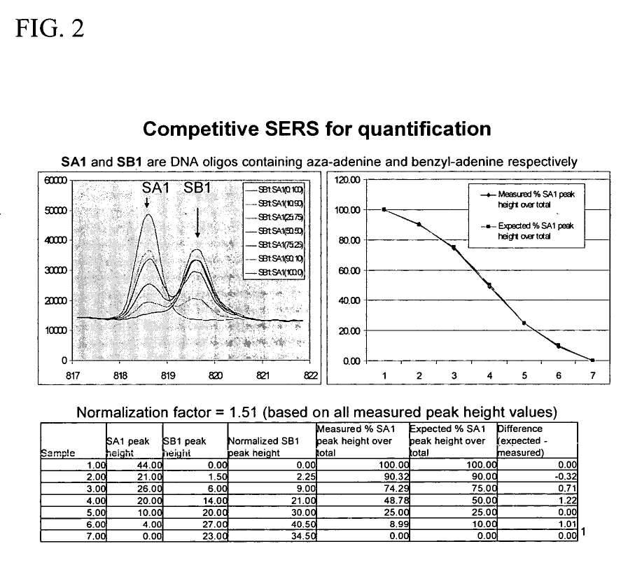

Once a series of Raman spectra are recorded for at least two analytes a normalization equation that correlates the Raman spectra of one analyte to another analyte can be prepared. For example, a normalization factor between data set 1 and data set 2=set 1 normalization factor (average ratio of expected over measured for data set 1) divide by set 2 normalization factor (average ratio of expected over measured for data set 2). See FIG. 2 for an example of the normalization factor.

The normalization equation allows for a very accurate concentration determination to be made when the concentration and identity of a first analyte is known and the identity, but not the concentration, of a second analyte is known. The concentration of the first analyte and the Raman spectra intensity of the first and second analytes are inserted into the normalization equation to determine the concentration of the second analyte. For example, to test the concentration of analyte 2 in a sample, a series of dilutions of the sample can be mixed with known concentrations of analyte 1; as a control a separate set of dilutions of known concentrations of analyte 1 and analyte 2 can be measured (or pre-measured and saved as a database or library).

FIG. 2 shows an example of how the normalization equation can be determined. In FIG. 2 SA1 and SB1 are DNA oligos containing aza-adenine and benzyl-adenine respectively. Raman spectra of the two DNA oligos were detected using a standard SERS method at various concentration ratios as indicated. Their peak heights were measured and a normalization factor was calculated based on the data.

The normalized peak heights matched well with the expected values (right graph) even though the absolute peak heights were not proportional to the concentration ratios (left graph).

Yet another embodiment, of this invention relates to taking into account alterations to an analyte's Raman spectra caused by other compounds that are found with an analyte. These other compounds can be, for example, contaminates or compounds that are used to solublize/suspend the analyte.

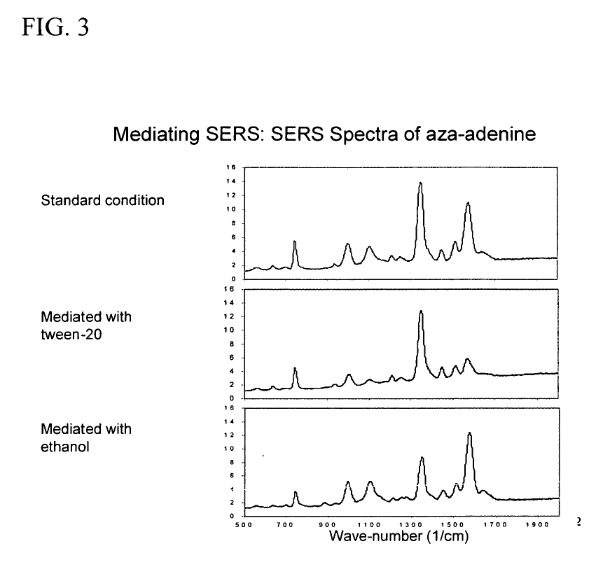

FIG. 3 shows the Raman specta of aza-adenine in the presence of three different mediating compounds. A mediating compound is an additive that is present along with the analyte in the SERS reaction solution. SERS signatures can be mediated by their solution environment. The Raman spectra (peak #, positions and peak heights) of an analyte are determined by the chemical composition, configuration and the analyte's interaction with the metal surface of the SERS active particles. Accordingly, mediating compounds can affect the Raman spectra of analyte by changing the composition of the sample being measured, the configuration of the analyte or how the analyte interacts with the surface of the SERS active particles.

In FIG. 3, 100 μL silver colloid including 4 μM 8-aza-adenine (AA) was mixed with 100 μL of a test reagent chosen from the following: water (standard condition), 1% TWEEN-20 (generically known as Polysorbate 20) (final 0.33% v/v), 100% ethanol (final 33%0.33% v/v) A resulting 200 μl mixture was then mixed with either 100 μL of 0.34 M LiCl before the Raman spectra of the samples were measured. Raman signal intensities were in arbitrary unit and normalized to respective maximums. As shown in FIG. 3, the aza-adenine SERS signal changes in response to different mediating agents (Polysorbate 20 and ethanol) in the solution. In these mediated interactions, there is no covalent modification.

The fact that different mediating agents that are present with the analyte can affect the Raman spectra of the analyte can be used to more accurately identify and measure the concentration of the analyte. For example, a library of one or more analytes in a variety of mediated environments can be produced and stored, for example in a computer memory. The Raman spectra of an unknown analyte in a known environment can then be more accurately predicted by taking into account changes to the Raman spectra that occurs because of the presence of the mediating environment. Alternatively, changes in the Raman spectra of an analyte because of its mediated environment can be used to identify/quantify which mediated compounds are present along with the analyte.

The mediating environment can be taken into account by comparing the Raman spectra of an unknown analyte to a library that includes analytes in mediating environments. If the mediating environment is known, the sample can be compared to spectrum for the known environment. Alternatively, if the environment is unknown, the analyte and/or the mediated environment can be determined by comparing the analyte to the library of analytes in a variety of mediated environments.

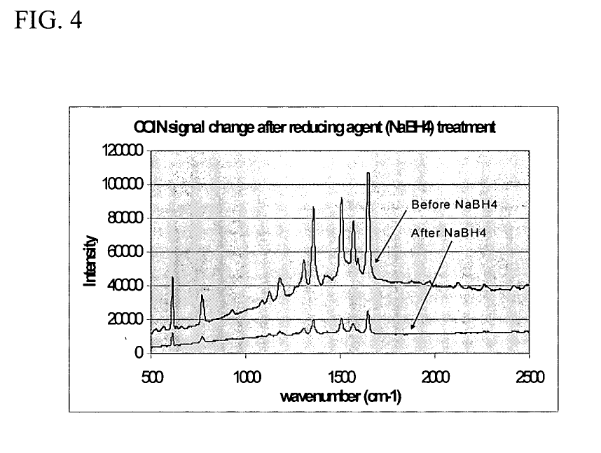

Another way that the mediating environment and/or the analyte itself can alter an analyte's Raman spectra is by interfering with the SERS active particles FIG. 4 shows how the Raman spectra of a COIN particle that includes a Raman active compound can be altered by reacting with the analyte or mediated environment. In FIG. 4 a rhodamine 6G (R6G) COIN signal is decreased due to reduction by NaBH₄. Again changes in the Raman signal due to interaction of the analyte or mediated environment can be taken into account by preparing a library that includes Raman spectra obtained in these environments and then comparing unknown analytes with the spectra in the library.

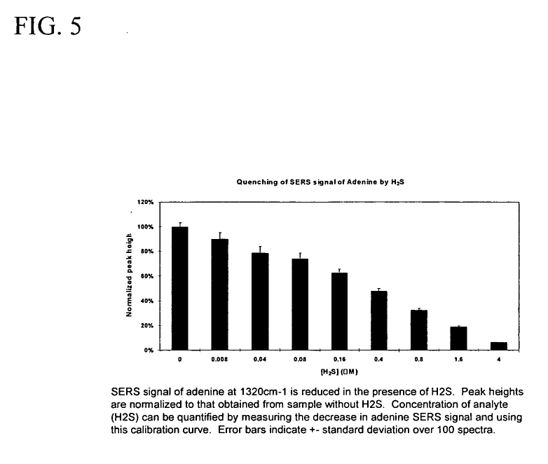

FIG. 5 shows how an analyte's interaction with a metal surface can also be used to quantify the amount of the analyte. In FIG. 5, the SERS signal of adenine at 1320 cm⁻¹ is reduced in the presence of H₂S. Peak heights are normalized to that obtained from a sample without H₂S. The concentration of the analyte H₂S is then quantified by measuring the decrease in the adenine SERS signal and using the calibration curve shown in FIG. 5. Error bars indicated±standard deviation over 100 spectra. Accordingly, adenine's SERS signal intensity reduction was proportional to the increasing quantity of H₂S (analyte) that modified SERS's silver particle surface. This same process of determining the decrease in Raman signal due to an analyte's interaction with the SERS particle surface can be used to quantify a variety of compounds that react with SERS particles.

Further, non-Raman-active analytes can also be identified/quantified by measuring their interaction with known Raman active compounds. For example, many organic compounds can interact with porphyrin molecules and change porphyrin SERS signals. The changes in these SERS signals can be used to identify and or quantify the organic compounds.

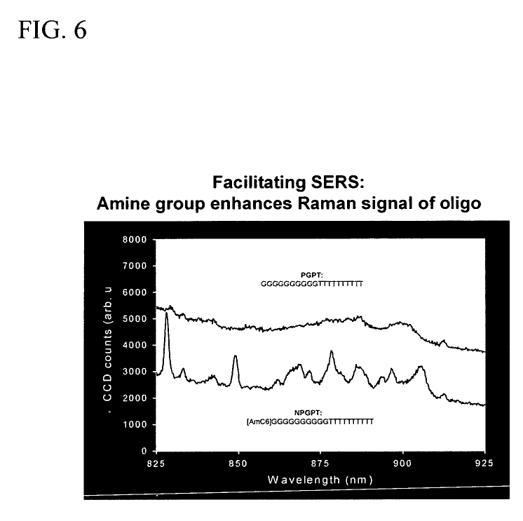

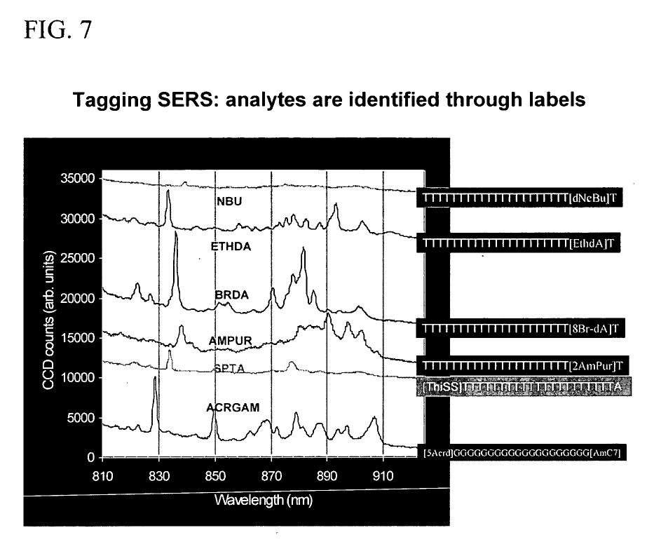

FIG. 6 shows that nucleic acid oligo (the analyte) can be measured by attaching amine group that enhances the Raman signals (facilitating SERS). FIG. 7 shows the tagging SERS method for analyte detection, where a label molecule can be attached to the analyte and the analyte is detected and identified through the label or tag.

This application discloses several numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1. A device comprising:
a substrate; and
a plurality of spots comprising surface enhanced Raman scattering (SERS) active particles attached to the substrate, wherein the SERS active particles generate surface enhanced Raman signal specific to the analyte molecules when the analyte-SERS complexes are excited with a light source.

2. The device of claim 1, wherein the SERS active particles comprise a metal.

3. The device of claim 1, wherein the SERS active particles comprise gold, silver, copper, lithium, sodium, potassium, palladium, platinum, or aluminum.

4. The device of claim 1, wherein the SERS active particles comprise composite-organic-inorganic-nanoparticles (COINs).

5. The device of claim 4, wherein the SERS active particles comprise gold, silver, platinum, copper, or aluminum.

6. The device of claim 4, wherein the SERS active particles comprise one or more compounds selected from the group consisting of adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, rhodamine 6G, rhodamine B, crystal violet, basic fuchsin, cyanine 2, cyanine 3, and 9-amino-acridine.

7. The device of claim 1, wherein the composition of the SERS active particles comprising a first spot has a different composition than the SERS active particles comprising at least one other spot.

8. The device of claim 1, wherein the concentration of the SERS active particles comprising a first spot have a different concentration than the SERS active particles comprising at least one other spot.

9. The device of claim 1, further comprising a Raman spectrometer.

10. The device of claim 1, wherein the substrate comprises a multiple-well array or a surface comprising a plurality of sub-surfaces.

11. The device of claim 2, wherein the SERS active particles are attached to the substrate through thiol groups.

12. A device comprising:
a substrate; and
a plurality of spots comprising composite-organic-inorganic-nanoparticles (COINs) attached to the substrate.

13. The device of claim 12, wherein the COINs comprise gold, silver, platinum, copper, or aluminum.

14. The device of claim 12, wherein the COINs comprise one or more compounds selected from the group consisting of adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, rhodamine 6G, rhodamine B, crystal violet, basic fuchsin, cyanine 2, cyanine 3, and 9-amino-acridine.

15. The device of claim 12 wherein the composition of the COINs comprising a first spot has a different composition than the COINs comprising at least one other spot.

16. The device of claim 12, wherein the concentration of the COINs comprising a first spot have a different concentration than the COINs comprising at least one other spot.

17. The device of claim 12, further comprising a Raman spectrometer.

18. The device of claim 12, wherein the substrate comprises a multiple-well array or a surface comprising a plurality of sub-surfaces.

19. The device of claim 12, wherein the COINs are attached to the substrate through bi-functional linker groups.

20. A method comprising:
determining a normalization equation that correlates a Raman spectra of a first analyte to a Raman spectra of a second analyte for a given analyte concentration;
obtaining the concentration of the first analyte in a mixture;
measuring the Raman spectra of the first analyte in the mixture and a Raman spectra of the second analyte in the mixture; and
determining the concentration of the second analyte in the mixture utilizing the normalization equation.

21. The method of claim 20, wherein determining the normalization equation comprises:
obtaining a Raman spectra of the first analyte across a range of concentration levels;
obtaining a Raman spectra of the second analyte across a range of concentrations; and
correlating the Raman spectra of the first analyte to the Raman spectra of the second analyte to determine the normalization equation.

22. The method of claim 20, wherein the Raman spectra of the first and second analytes in the mixture are obtained utilizing composite-organic-inorganic-nanoparticles (COINs).

23. The method of claim 20, wherein the Raman spectra of the first and second analytes in the mixture are obtained utilizing surface enhanced Raman scattering (SERS) active particles.

24. The method of claim 23, wherein the SERS active particles comprise gold, silver, copper, lithium, sodium, potassium, palladium, platinum, or aluminum.

25. The method of claim 20, wherein the first analyte and the second analyte are related in terms of molecular backbone structure, with difference in side groups or difference in configuration of the side groups with respect to the backbone structure.

26. The method of claim 20, wherein the normalization equation is linear for a range of concentrations.

27. The method of claim 20, wherein the normalization equation is non-linear for a range of concentrations.

28. The method of claim 20, wherein the first and second analytes are both organic compounds.

29. The method of claim 20, wherein the first and second analytes are both inorganic compounds.

30. The method of claim 20, wherein Raman active labels are attached to the first and second analytes.

31. The method of claim 20, wherein the mixture comprises a third analyte.

32. The method of claim 20, wherein the Raman spectra of the first analyte is created by modifying the Raman spectra of a Raman active compound.

33. The method of claim 20, wherein the first analyte is not a Raman active compound and the Raman spectra of the first analyte is created by modifying the Raman spectra of a Raman active compound.

34. A method comprising:
obtaining a Raman spectra of a series of known substances in one or more environments;
measuring a Raman spectra of an analyte in a mixture; and
comparing the Raman spectra of the analyte in the mixture to the Raman spectra of the series of known substances in one or more environments to identify the analyte in the mixture.

35. The method of claim 34, wherein the Raman spectra of the series of known substances in one or more environments are obtained by measuring the Raman spectra of the known substances in one or more environments.

36. The method of claim 34, wherein the mixture comprises water, ethanol or polysorbate 20.

37. The method of claim 34, wherein the mixture comprises water.

38. The method of claim 34, wherein the mixture comprises a reducing agent.

39. The method of claim 34, wherein one or more components in the mixture reacts with the analyte.

40. The method of claim 34, wherein the Raman spectra of the analyte in the mixture is measured using surface enhanced Raman scattering (SERS) active particles.

41. The method of claim 34, wherein the SERS active particles comprise particles comprise gold, silver, copper, lithium, sodium, potassium, palladium, platinum, or aluminum.

42. The method of claim 40, wherein one or more components of the mixture reacts with the SERS active particles.

43. The method of claim 40, wherein the SERS active particles comprise composite-organic-inorganic-nanoparticles (COINs).

44. The method of claim 34, further comprising identifying one or more components in the mixture besides the analyte.

45. The method of claim 34, wherein the Raman spectra of the series of known substances in one or more environments are obtained for a plurality of known substance concentrations.

46. A method comprising:
obtaining a Raman spectra of a known substance in one or more environments at a plurality of different concentrations;
measuring a Raman spectra of an analyte in a mixture; and
comparing the Raman spectra of the analyte in the mixture to the Raman spectra of a known substance in one or more environments at a plurality of different concentrations to determine the concentration of the analyte in the mixture.

47. The method of claim 46, wherein the Raman spectra of a known substance in one or more environments at a plurality of different concentrations are obtained by measuring the Raman spectra of the known substance in one or more environments.

48. The method of claim 46, wherein the mixture comprises water, ethanol or polysorbate 20.

49. The method of claim 46, wherein mixture comprises water.

50. The method of claim 46, wherein the mixture comprises a reducing agent.

51. The method of claim 46, wherein one or more components in the mixture reacts with the analyte.

52. The method of claim 46, wherein the Raman spectra of the analyte in the mixture is measured using surface enhanced Raman scattering (SERS) active particles.

53. The method of claim 46, wherein the SERS active particles comprise particles comprise gold, silver, copper, lithium, sodium, potassium, palladium, platinum, or aluminum.

54. The method of claim 52, wherein one or more components of the mixture reacts with the SERS active particles.

55. The method of claim 52, wherein the SERS active particles comprise composite-organic-inorganic-nanoparticles (COINs).

56. The method of claim 46, further comprising identifying one or more components in the mixture besides the analyte.

57. The method of claim 46, wherein the Raman spectra of the series of known substances in one or more environments are obtained for a plurality of known substances.
